(19) United States
(12) Patent Application Publication
(10) Pub. No.: US 2024/0207174 A1
(43) Pub. Date: Jun. 27, 2024

(54) DISSOLVABLE POLYMERIC EYE INSERTS

(71) Applicant: Alcon Inc., Fribourg (CH)

(72) Inventors: Adam C. Reboul, Roswell, GA (US); Katily Ramirez, Pembroke Pines, FL (US); Ye Hong, Flower Mound, TX (US); Qiaozhi Lu, Fort Worth, TX (US)

(21) Appl. No.: 18/536,437

(22) Filed: Dec. 12, 2023

Related U.S. Application Data
(60) Provisional application No. 63/477,112, filed on Dec. 23, 2022.

(51) Int. Cl. A61K 9/00; A61K 31/08; A61K 31/225; A61K 31/355; A61K 47/32; A61K 47/36

(52) U.S. Cl. CPC A61K 9/0051; A61K 31/08; A61K 31/225; A61K 31/355; A61K 47/32; A61K 47/36

(57) ABSTRACT

Polymeric eye inserts are provided that may be dissolvable when placed in the cul-de-sac of the eye. The polymeric eye insert comprises at least one monovalent alginate layer and at least one multivalent salt layer. The at least one monovalent alginate layer is substantially free of the multivalent salt, and the at least multivalent salt layer is substantially free of the monovalent alginate.

The present disclosure generally relates to polymeric eye insert technology, and more particularly to dissolvable polymeric eye inserts having a monovalent alginate and a multivalent salt and the eye inserts release humectants and drugs into the eye (including, but not limited to the anterior and posterior segments) for an extended duration of time compared to topical drop dosage forms.

### BACKGROUND

Many ophthalmic formulations comprise compounds that provide lubricity and other desirable properties. When these formulations are instilled in the eye, the properties of such compounds can prevent undesirable problems such as bioadhesion and the formation of friction-induced tissue damage, as well as encourage the natural healing and restoration of previously damaged tissues.

Compliance with administration of topically applied ophthalmic formulations such as liquids, ointments, gels, sprays is often poor, specifically for the treatment of dry eye, allergy, infection and slowly progressing diseases, such as glaucoma, requiring multiple applications per day to lubricate and deliver a drug to the eye. Exposure to topically administered aqueous formulations is often driven by the short retention time of the formulation on the ocular surface, which can be less than 25 minutes following instillation. Paugh et al., Optom Vis Sci. 2008 August; 85(8):725-31. Typical aqueous formulations for ocular use may be diluted or washed from the ocular surface within minutes, introduce variability in the usage, or result in less accurate and precise dosages administered to the eye. Accordingly, there is a need to reduce treatment burden and improve compliance.

Ointments and gels, which are highly viscous and usually reside in the eye longer than a liquid can provide for more accurate administration. However, they can also interfere with a patient's vision and may require, at a minimum, dosing 2-3 times per day. For these and other reasons the rate of discontinuation of use can be very high. Swanson, M., J. Am. Optom. Assoc., 2011; 10:649-6.

Inserts, both bioerodible and non-bioerodible, are also available and allow for less frequent administration. Insert can continuous delivery of lubricants or drugs offers advantages over the conventional ocular therapies that involve administration of lubricants or drugs solutions or suspensions as eye drops. Pescina S et al., Drug Dev Ind Pharm; 2017 May 7:1-8; Karthikeyan, M B et al., Asian J. Pharmacol; 2008; October-Decemcer 192-200. These inserts, however, require complex and detailed preparation and can be uncomfortable to the patient. An additional problem with non-bioerodible inserts is that they must be removed after use. However, with proper use and adequate patient education, inserts can be an effective and safe treatment choice for dry eye patients.

Hydroxypropyl cellulose ophthalmic inserts such as LACRISERT® (Aton Pharmaceuticals Inc.) have been used for dry eye patients. These inserts are translucent cellulose-based rods measuring 1.27 mm in diameter and 3.5 mm in length. Each of these inserts contains 5 mg of hydroxypropyl cellulose, with no preservatives or other ingredients. The medication is administered by placing a single insert into the inferior cul-de-sac of the eye beneath the base of the tarsus. These inserts are indicated particularly for patients who continue to have dry eye symptoms following an adequate trial therapy with artificial tears. They also are indicated for patients with keratoconjunctivitis sicca, exposure keratitis, decreased corneal sensitivity, and recurrent corneal erosions. Several studies have been performed to evaluate the efficacy of HPC ophthalmic inserts. (Luchs, J, et al., Cornea, 2010; 29:1417-1427; Koffler B, et al., Eye Contact Lens; 2010; 36:170-176; McDonald M, et al., Trans Am Ophthalmol. Soc., 2009; 107:214-221; Wander A, and Koffler B, Ocul Surf. 2009 July; 7(3):154-62).

However, there also are challenges in using these types of inserts. For example, LACRISERT® inserts tend to dissolve slowly and can remain in the eye even after 15-20 hours. The rod is hard and inelastic with edges due to rod-shaped design. The slow dissolving properties coupled with the rod hardness and design may lead to side effects including blurred vision, foreign body sensation and/or discomfort, ocular irritation or hyperemia, hypersensitivity, photophobia, eyelid edema, and caking or drying of viscous material on eyelashes. The most common side effect of these hydroxypropyl cellulose ophthalmic inserts is blurred vision due to the long retention time of the insert.

Thus, there is a need to develop polymeric eye inserts that are comfortable, controlled dissolution rate to release lubricants and drugs and improve patient compliance.

### SUMMARY

The invention provides A polymeric eye insert, the insert comprising:
a) 5% to 50% w/w gelling agent monovalent alginate, b) one or more humectant, c) one or more mucoadhesive polymers that are biocompatible with the ocular surface and tear film of the eye and d) 0.1% to 5% w/w multivalent salt, wherein the insert is prepared by a co-extrusion process, wherein polymeric eye insert comprising at least one monovalent alginate layer and at least one multivalent salt layer, wherein the at least one monovalent alginate layer is substantially free of the multi valent salt, wherein the at least one multivalent salt layer is substantially free of the monovalent alginate.

The present invention is partly based on the finding that adding monovalent alginate (for example, sodium alginate) and a multivalent salt to a polymeric eye insert of mucoadhesive polymers that are biocompatible with the ocular surface and tear film of the eye can have a longer dissolution time of the polymeric eye insert. The sodium alginate containing eye insert can have a longer dissolution time on the ocular surface because di-valent or higher valent salt (for example a calcium chloride) forms gel with sodium alginate.

The present invention is surprisingly finding that multivalent alginate (for example, calcium alginate) gel formed by crosslinking between multivalent cation and anionic alginate.is water insoluble but slowly dissolved in tear film of the eye. Without intending to be limited by theory, the sodium ions of present in the tear fluid slowly break down and disintegrate the calcium alginate gel by displacing the calcium of the calcium alginate and forming sodium alginate which is water soluble. The dissolving of the multi valent alginate (for example, calcium alginate) depends on the removal of calcium from the gelling blocks of alginate by substitution with sodium of the tear, which results this cation substitution, is water soluble and therefore, calcium alginate dissolves.

The present invention is partly based on the finding that the typical eye inserts formed by compression molding and solution casting are non-homogeneous and have lumps of gels formed by sodium alginate and calcium chloride if both are present in the eye insert. The present invention is partly based on the finding that the problems of non-homogeneous and lumps of gels are reduced significantly.

The present invention is partly based on the finding that polymeric eye inserts formed by co-extrusion process to form the eye inserts having multiple layers and separating sodium alginate and calcium chloride in two different layers on different layers to avoid the problem of non-homogeneous and having lumps of gels formed by sodium alginate and calcium chloride. When the polymeric eye insert of the present invention is put on the eye, the multiple-layer polymeric eye insert partially dissolves and release cationic calcium and anionic alginate from separated layers of the insert. The released cationic calcium and anionic alginate form gels in situ to sustain the retention time of the polymeric eye inserts on the eye as well as sustain the release of drugs from the polymeric eye insert.

The polymeric eye insert may reduce dosing frequency and patient burden typically associated with topical drop usage. These polymeric eye inserts also may include one or more pharmaceutically active agents.

### BRIEF DESCRIPTION OF THE DRAWINGS

FIG. 1 is a diagram illustrating the manufacture of a co-extruded eye insert according to the invention.

FIG. 2 is a diagram illustrating the manufacture of a co-extruded eye insert according to the invention.

FIG. 3 is a diagram showing a co-extruded two-layer eye insert according to the invention.

FIG. 4 is a diagram showing a co-extruded three-layer eye insert according to the invention.

### DETAILED DESCRIPTION

Unless defined otherwise, all technical and scientific terms used herein have the same meaning as commonly understood by one of ordinary skill in the art to which this invention belongs. Generally, the nomenclature used herein, and the laboratory procedures are well known and commonly employed in the art. Conventional methods are used for these procedures, such as those provided in the art and various general references. Where a term is provided in the singular, the inventors also contemplate the plural of that term. The nomenclature used herein, and the laboratory procedures described below are those well-known and commonly employed in the art.

“About” as used herein means that a number referred to as “about” comprises the recited number plus or minus 1-10% of that recited number.

“Monovalent alginate” as used herein means sodium alginate, potassium alginate, cesium alginate or lithium alginate.

As used in this application, “dissolution time” refers to the time taken for the insert to completely dissolve i.e., break down from a solid gel like material in a vehicle under specified condition to form a homogeneous solution. The dissolution time is measured by the procedure described here: 6 mm diameter film disks were cut and placed at the bottom of the well with isotonic saline with methyl blue. The well was placed in orbital shaker and shaken until the insert had dissolved (complete disintegration by visual inspection. The dissolution time was recorded.

The term “multivalent salt” refers to a salt contains a di- or higher valent cation including Ca²⁺, Fe²⁺, Fe³⁺, Ag²⁺, Ag³⁺, Au²⁺, Au³⁺, Mg²⁺, Cu²⁺, Cu³⁺, Zn²⁺ or ammonium-In one embodiment of the invention, the cation is Ca²⁺. While not wishing to be bound by theory or explanation, it is believed that the exposure to the multivalent salt forms a gel network with the monovalent alginate (for example, sodium alginate) that provides a longer dissociation time than the eye insert containing no multivalent salt.

“Extrusion” as used herein means a process where a material undergoes plastic deformation by the application of a force causing that material to flow through an orifice or die. The material adopts the cross-sectional profile of the die and if the material has suitable properties, that shape is retained in the final extrudate. According to the present application, Extrusion processes include single screw extrusion, twin screw extrusion, co-extrusion.

Single screw extrusion uses one screw within a cylindrical barrel to continuously push plastic through a constant profile die.

A twin-screw extrusion uses two intermeshing, co-rotating screws mounted on splined shafts in a closed barrel. The screws are tight and self-wiping, which eliminates stagnant zones over the entire length of the process section. This results in high efficiency and perfect self-cleaning

“Co-extrusion” and “co-extruded sheet” as used herein means a product that has two or more layers made by extruding two or more materials through a single die with two or more orifices arranged so that the extrudates merge and weld together into a laminar structure. The invention is directed to co-extrusions in general. Although existing technology requires co-extrusions to be made in a sheet the invention is not so limited.

Multi-layer extrusion technology is a process in which two or more polymers are extruded and simultaneously joined in a co-extrusion die head to form tubing with multiple layers

“Multivalent salt layer” as used herein means a layer that comprises di- or higher valent salt and is substantially free of monovalent alginate.
“Monovalent alginate layer” as used herein means a layer that comprises monovalent alginate and is substantially free of multivalent salt.
According to the present invention, the eye insert is substantially free of a monovalent alginate, or a multivalent salt refers to be less than 0.005% W/W monovalent alginate or multivalent salt, preferably less than 0.002% W/W monovalent alginate or multivalent salt, respectively.

In an embodiment of the present disclosure, a polymeric eye insert may be comprised of a gel agent such as sodium alginate, hyaluronic acid, hydroxypropyl guar (HP guar), and at least one humectant, such as polyethylene glycol (PEG) or polyvinylpyrrolidone (PVP) or combinations thereof. However, other polymers may be used without departing from the present disclosure, as described herein. An insert according to embodiments of the present disclosure may be inserted in the lower eye lid (also known as the cul-de-sac) of the eye, and upon insertion, the insert may absorb tears and dissolve to lubricate with the composition and protect the ocular surface for an extended duration superior to previously known topical ophthalmic compositions. Pharmaceutically active agents also may be incorporated into polymeric eye inserts according to embodiments of the present disclosure. Insertion of a polymeric eye insert according to embodiments of the present disclosure may provide relief to the patient from symptoms of dry eye as well as other eye conditions.

The biomaterial for forming a polymeric eye insert according to embodiments of the present disclosure may be comprised of one or more mucoadhesive polymers that are biocompatible with the ocular surface and tear film. Mucoadhesive polymers have numerous hydrophilic groups, such as hydroxyl, carboxyl, amide, and sulfate. These groups attach to mucus or the cell membrane by various interactions such as hydrogen bonding and hydrophobic or electrostatic interactions. mucoadhesive polymers serves as tear volume supplementation, tear film stabilization, and protection of the ocular surface by reducing friction between the eyelids and the cornea. Thus, mucoadhesive polymers are effective ocular surface lubricants.

Polymers that may be used in polymeric eye inserts according to embodiments of the present disclosure include, but are not limited to, hyaluronic acid (in acid or salt form), hydroxypropyl methylcellulose (HPMC), methylcellulose, tamarind seed polysaccharide (TSP), G al a c t o m a n n a n s, f o r e x a m p l e s; guar and derivatives thereof such as hydroxypropyl guar (HP guar), scleroglucan poloxamer, poly(galacturonic) acid, pectin, xanthan gum, xyloglucan gum, chitosan, sodium carboxymethylcellulose, polyvinyl alcohol, carbomer, polyacrylic acid and/or combinations thereof.

The preferred biocompatible mucoadhesive polymers are hyaluronic acid, guar, and derivatives and/or combinations thereof. Hyaluronic acid is an unsulphated glycosaminoglycan composed of repeating disaccharide units of N-acetylglucosamine (GlcNAc) and glucuronic acid (GlcUA) linked together by alternating beta-1,4 and beta-1,3 glycosidic bonds. Hyaluronic acid is anionic nature polymer and also known as hyaluronan, hyaluronate, or HA. As used herein, the term hyaluronic acid also includes salt forms of hyaluronic acid such as sodium hyaluronate. A preferred hyaluronic acid is sodium hyaluronate. The weight average molecular weight of the hyaluronic acid used in insert of the present invention may vary but is typically weight average molecular weight of 0.1 to 3.0M Daltons. In one embodiment, the hyaluronic acid has a weight average molecular weight of 0.5 to 2.0MDaltons. In another embodiment, the hyaluronic acid has a weight average molecular weight of 1.5 to 2.0 M Daltons.

According to the present invention, the hyaluronic acid is present in an amount of from about 5% to 55% by weight, or from about 15% to 50% by weight, or from about 20% to 45% by weight, or from about 30% to 40% by weight, or from about 20% to 30% by weight based on the total weight of the composition, including increments and ranges thereof and therebetween.

As used in this application, a galactomannan polymer refers to a galactomannan (e.g., guar) and/or a chemically modified galactomannan.

A galactomannan, as known to a person skilled in the art, is a polysaccharide consisting of a mannose backbone with galactose side groups (more specifically, a (1-4)-linked beta-D-mannopyranose backbone with branch points from their 6-positions linked to alpha-D-galactose, (i.e., 1-6-linked alpha-D-galactopyranose). The ratio of D-galactose to D-mannose in galactomannan can vary, but generally will be from about 1:2 to 1:4. Galactomannans having a D-galactose:D-mannose ratio of about 1:2 is most preferred. Preferred galactomannan is guar.

Galactomannans may be obtained from numerous sources. Such sources include guar gum, locust bean gum and tara gum, as further described below.

Guar gum is the ground endosperm of Cyamopisis tetragonolobus (L.) Taub. The water-soluble fraction (85%) is called “guaran” (molecular weight of 220,000), which consists of linear chains of (1-4)-β-D mannopyranosyl units with α-D-galactopyranosyl units attached by (1-6) linkages. The ratio of D-galactose to D-mannose in guaran is about 1:2. The gum has been cultivated in Asia for centuries and is primarily used in food and personal care products for its thickening property. It has five to eight times the thickening power of starch. Guar gum may be obtained, for example, from Rhone-Polulenc (Cranbury, N.J.), Hercules, Inc. (Wilmington, Del.) and TIC Gum, Inc. (Belcamp, Md.).

Locust bean gum or carob bean gum is the refined endosperm of the seed of the carob tree, Ceratonia siliqua. The ratio of galactose to mannose for this type of gum is about 1:4. Cultivation of the carob tree is old and well known in the art. This type of gum is commercially available and may be obtained from TIC Gum, Inc. (Bekamp, Md.) and Rhone-Polulenc (Cranbury, N.J.).

Tara gum is derived from the refined seed gum of the tara tree. The ratio of galactose to mannose is about 1:3. Tara gum is not produced in the United States commercially, but the gum may be obtained from various sources outside the United States.

A chemically modified galactomannan is a derivative of a galactomannan in which some (but not all) of hydrogen atoms of the hydroxyl groups are substituted with an organic group. Examples of preferred chemically-modified glactomannans includes without limitation hydroxyethyl-substituted galactomannan (e.g., hydroxyethyl guar), hydroxypropyl galactomannan (e.g., hydroxypropyl guar), C₁-C₃ alkyl galactomannan (e.g., methyl guar, ethyl guar, propyl guar), carboxymethyl galactomannan (e.g., carboxymethyl guar), carboxymethylhydroxypropyl galactomannan (e.g., carboxymethylhydroxypropyl guar), hydroxypropyltrimonium chloride galactomannan (e.g., hydroxypropyltrimonium chloride guar), and combinations thereof. Preferred chemically-modified glactomannans are hydroxypropyl guar.

Hydroxyethyl guar, hydroxypropyl guar, methyl guar, ethyl guar, propyl guar, carboxymethyl guar, carboxymethylhydroxypropyl guar, and hydroxypropyltrimonium chloride guar are well known and are commercially available. For example, modified galactomannans of various degree of substitution are commercially available from Rhone-Poulenc (Cranbury, N.J.).

Preferred galactomannans of the present invention are guar and hydroxypropyl guar. Hydroxypropyl guar is particularly preferred. The weight average molecular weight of the Hydroxypropyl guar in the insert of the present invention may vary but is typically 0.5 to 5 M Daltons. In one embodiment, the Hydroxypropyl guar has a weight average molecular weight of 2 to 4 M Daltons. In another embodiment, the Hydroxypropyl guar has a weight average molecular weight of 2 to 3 M Daltons.

According to the present invention, the Hydroxypropyl guar is present in an amount of from about 5% to 55% by weight, or from about 15% to 50% by weight, or from about 20% to 45% by weight, or from about 30% to 40% by weight based on the total weight of the composition, including increments and ranges thereof and therebetween.

Polymers used in inserts according to embodiments of the present disclosure should be non-toxic and able to solubilize in eye fluids to ensure that the insert is eventually dissolved, generally over a 120-minute time but less than 480 minutes frame. It should be appreciated that the polymer(s) selected should be mucoadhesive. It also should be appreciated that one or more polymers may be blended according to embodiments of the present disclosure. For example, in an embodiment of the present disclosure, hyaluronic acid (HA) may be blended with tamarind seed polysaccharide (TSP) because TSP has been shown to increase residence time of HA in aggregate blends and the blend has desired film mechanical and lubrication properties.

In other embodiments of the present disclosure, as described in further detail below, hyaluronic acid may be combined with HP guar.

In another embodiment of the present disclosure, the polymeric eye insert further comprises a sodium alginate as a gelling agent to increase the dissolution time of the polymeric eye insert by including a multivalent salt. The multivalent salt is a salt containing a di- or higher valent. Examples of multivalent salt include, but not limited to Ca²⁺, Fe²⁺, Fe³⁺, Ag²⁺, Ag³⁺, Au²⁺, Au³⁺, Mg²⁺, Cu²⁺, Cu³⁺, Zn²⁺ or ammonium. As it is commonly known in the art, and as used herein, the alginate compounds are polysaccharides which are formed from units of beta-1,4-D-mannuronic acid and alpha-1,4-L-guluronic acid. The units of the alginate compound may be arranged in any manner, i.e., in random or block arrangement. A gel composed of three-dimensional network is formed upon gelation process of alginate when blocks of guluronic acid residues interact ionically with multivalent cation such as Ca²⁺. Preferably, the alginate compound is low in mannuronic acid units relative to guluronic acid units. Specifically, the ratio (by number of units, not by weight of units) of mannuronic acid units to guluronic acid units is preferably less than about 1, more preferably from about 0.1 to about 0.9, and most preferably from about 0.1 to about 0.5.

Any alginate compound may be utilized in the compositions of the present invention. For example, the alginate compound may be a naturally occurring alginate compound (naturally occurring alginates may, for example, be derived from seaweed). As used herein, the term “naturally occurring” with respect to the alginate compound means that the alginate compound utilized is found in nature or is prepared synthetically, but chemically equivalent to an alginate compound found in nature. Other alginate compounds which may be utilized include those which are derivatives of naturally occurring alginates, for example, a propylene glycol alginate. Preferably, the alginate compound utilized herein is a naturally occurring alginate.

A gel composed of three-dimensional network is formed upon gelation process of alginate when blocks of guluronic acid residues interact ionically with multivalent cation such as Ca²⁺. Preferably, the alginate compound is low in mannuronic acid units relative to guluronic acid units. Specifically, the ratio (by number of units, not by weight of units) of mannuronic acid units to guluronic acid units is preferably less than about 1, more preferably from about 0.1 to about 0.9, and most preferably from about 0.1 to about 0.5.

According to the present application, any kind of monovalent alginate can be used. A preferred alginate compound for use in the present compositions is sodium alginate. Sodium alginate is commercially available from a variety of sources including, for example, NutraSweet Kelco Company supplies numerous alginate compounds including, for example, those in the KELGIN series, MANUCOL series, KELVIS series, KELCOSOL series, KELTONE series, MANUGEL series, KELMAR series, KELCOLOID series, KELSET series, LACTICOL series, ALGINADE series, DARILOID series, MARLOID series, and SHERBELIZER series. Thermo Scientific™ supplies numerous alginate compounds including, for example, those in Alginic acid sodium salt, high viscosity, low viscosity, and very low viscosity.

Alginate is reported to be generally non-toxic, biodegradable, non-immunogenic and biocompatible. Sodium alginate is the salt of alginic acid. When small drops of sodium alginate solution are dropped into calcium chloride solution a cation exchange between Na⁺ and Ca²⁺ takes place and a gel is obtained. The alginate gel can be used in drug controlled release. Alginates are among the most widely used biopolymers in the field of pharmaceutics. They are conventionally used as an excipient in drug products due to their thickening, gel-forming, and stabilizing properties. Alginate salt solution can form a gel in the presence of multivalent ions such as Ca²⁺, Fe²⁺, Fe³⁺, Ag²⁺, Ag³⁺, Au²⁺, Au³⁺, Mg²⁺Cu²⁺, Cu³⁺, and Zn²⁺, which can be utilized to incorporate numerous drugs, proteins, cells, or enzymes that can be released in a controlled manner. Therefore, alginate can play a significant role in the design of a tailored controlled-release product.

The sodium alginate compound present in the composition according to the invention is present in an amount of from about 10% to 50% by weight, or from about 12% to 45% by weight, or from about 15% to 40% by weight, or from about 17% to 35% by weight, or from about 20% to 30% by weight based on the total weight of the composition, including increments and ranges thereof and therebetween. The weight average molecular weight of the sodium alginate used in insert of the present invention may vary but is typically weight average molecular weight of 0.5 to 3M Daltons or 1 to 2.5M Daltons. In one embodiment, the sodium alginate has a weight average molecular weight of 1.5 to 2.5 M Daltons. In another embodiment, the sodium alginate has a weight average molecular weight of 2 to 2.5 M Daltons.

In some embodiments, the multivalent salt is present in an amount of from about 0.1% to about 5% w/w, about 0.2% to about 4% w/w, about 0.3% to about 3% w/w dry weight of the polymeric eye insert.

In some embodiments, the one or more mucoadhesive polymers are present in an amount of from about 10% to about 90% w/w, about 50% to about 80% w/w, about 60% to about 70% w/w, or about 50% to about 60% w/w by dry weight of the polymeric eye insert, provided that the sum of the % w/w of mucoadhesive polymers, % w/w of the sodium alginate, % w/w of the humectant agents and other components may not listed above is 100% w/w.

The overall dry weight or mass of the polymeric eye insert may be in the range of about 0.5 to about 10 mg, or about 1 to about 5 mg, and in particular embodiments may be from about 1.5 to about 2.5 mg.

In some embodiments of the present disclosure, at least one humectant may be added to the one or more polymers to facilitate fabrication of a moisturizer delivery system and provide improved lubrication, hydration, and comfort upon insertion. It should be appreciated that humectant may be low or high-molecular weight compounds, including not limited to, polyethylene glycol (PEG) and derivatives thereof, propylene glycol, glycerin, and polyvinylpyrrolidone (PVP).

It is generally polyvinylpyrrolidone (PVP).is characterized by its viscosity in aqueous solution relative to that of water and expressed as a K value in the range of 10-120. For example, PVP K15, K30, 60, 90, 120 has a molecular weight of 8K, 60K, 400 k, 1.3 M and, 3M Daltons, respectively. The weight average molecular weight of the PVP used in insert of the present invention may vary but is typically weight average molecular weight of 60K to 3M Daltons. In one embodiment, the PVP has a weight average molecular weight of 400K to 2.5 M Daltons. In another embodiment, the PVP has a weight average molecular weight of 1 to 1.5 M Daltons. The weight average molecular weight of the PEG used in insert of the present invention is typically less than 20K Daltons and may vary but is typically weight average molecular weight of 200 to 9000 Daltons. In one embodiment, the PEG has a weight average molecular weight of 300 to 1000 Daltons. In another embodiment, the PEG has a weight average molecular weight of 350 to 450 Daltons.

In some embodiments, the humectant is present in an amount of from about 1% to about 10% w/w, about 1.5% to about 5% w/w, about 1.75% to about 3% w/w, or about 2% to about 2.5% w/w by dry weight of the polymeric eye insert, provided that the sum of the % w/w of mucoadhesive polymers, sodium alginate and % w/w of the humectant and other components not listed above is 100% w/w.

According to the present invention, polyethylene glycol (PEG) and polyvinylpyrrolidone (PVP) are defined as humectant agent, a sodium alginate as a gelling agent. In the present invention, polyethylene glycol (PEG) and polyvinylpyrrolidone (PVP) is primarily used as a humectant agent to facilitate fabrication of a moisturizer delivery system and provide improved lubrication, hydration, and comfort upon insertion even PEG and PVP may also have other functions. In the present invention, a sodium alginate is primarily used as a gelling agent to form a gelling network when contacting with a divalent metal cation even though sodium alginate may also have other functions.

The overall dry weight or mass of the ocular insert may be in the range of about 0.5 to about 10 mg, or about 1 to about 5 mg, and in particular embodiments may be from about 1.5 to about 2.5 mg.

The polymeric eye insert may be of any size or shape suitable for administration to the eye. Exemplary shapes include film, a rod, a sphere, or an irregular shape.

The insert also has a thickness and elasticity that is relatively comfortable for the user. A preferred thickness is between 25-250 microns, and a most preferred thickness is between 30-80 microns. The target thickness is 30 to 50 microns for films dissolving in more than 2 hours but less than 48 hours, preferred less than 24 hours.

In some embodiments, the ocular insert of the invention can have an on-eye dissolution time of at least 3 hours, preferably at least 4 hours, more preferably from about 4 to 48 hours. The on-eye dissolution time can be determined by determining the dissolution time of an ocular insert in saline or PBS solution know to a person skilled in the art. The dissolution time of an ocular insert of the invention can be fine-tuned by selecting amount of sodium alginate which can be activated by a salt activator such as calcium chloride for crosslinking to form a divalent metal iron alginate gelling network when a formed ocular insert is ready to be used.

According to the present application, the concentration of salt activator such as calcium chloride has a range from 0.1 gram/liter to 3 gram/liter, 0.3 gram/liter to 2.5 gram/liter, and 0.5 gram/liter to 2 gram/liter.

In some embodiments of the present disclosure, the polymeric eye insert does not include an additional pharmaceutically active agent. In other embodiments, the polymeric eye insert may include one or more additional pharmaceutically active agents. In some embodiments, the one or more pharmaceutically active agents may be selected from the group of antioxidant, vitamins ocular lubricants, anti-redness relievers such as brimonidine tartrate, tetrahydrozoline, naphazoline, cooling agents such as menthol, steroids and nonsteroidal anti-inflammatory agents to relieve ocular pain and inflammation, antibiotics, anti-histamines such as olopatadine, anti-virals, antibiotics and anti-bacterials for infectious conjunctivitis, anti-muscarinics such as atropine and derivatives thereof for myopia treatment, and glaucoma drug delivery such as prostaglandin and prostaglandin analogs such as travoprost, or therapeutically suitable combinations thereof.

Polymeric eye inserts according to embodiments of the present disclosure may be made using various processing techniques, including but not limited to, compression molding and solution casting. Compression molding may be carried out at temperatures and pressures that do not change the material or lead to significant side reactions. For example, compression molding of partially hydrated polysaccharides may use a compressional force of approximately 5,000-12,000 pounds at approximately 200-300 degrees Celsius for approximately 1-2 minutes. Solution or film casting may be carried out using solvents and/or co-solvents that may provide homogeneous films with little to no defects. The solvent may be removed by air or vacuum drying, resulting in an insert material that may be free from residual solvents. For example, a 1% (w/v) aqueous solution of polymer (or blend) may be cast and then allowed to evaporate. The film may then be cut with an oval-shaped punch of desired size and geometry. In some cases, the eye inserts formed by compression molding and solution casting are non-homogeneous and have lumps of gels formed by sodium alginate and calcium chloride. The problems of non-homogeneous and lumps of gels are reduced by using single extrusion or twin extrusion process to manufacture the eye inserts. Furthermore, the problems of non-homogeneous and lumps of gels are avoided by using co-extrusion process to form the eye inserts having multiple layers and separating sodium alginate and calcium chloride in two different layers on different layers. According to the present invention, the at least one monovalent alginate layer is substantially free of the multivalent salt, and the at least multivalent salt layer is substantially free of the monovalent alginate.

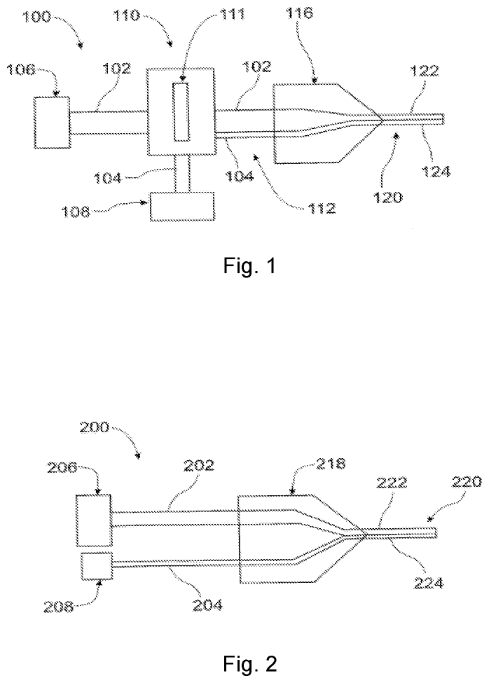

FIG. 1 illustrates feed-block co-extrusion 100, which is one suitable process for making a co-extruded sheet 120 according to the invention.

As shown in FIG. 1 sodium alginate layer material feed 102 from main extruder 106 and calcium chloride layer material feed 104 from the co-extruder 108 enter feed block 110 where they are aligned and proceed as a combined melt stream 112 to single manifold extrusion die 116 before being co-extruded as co-extruded sheet 120. In the feedblock 110 there is a plug 111 that determines how the two molten polymers 102, 104 will be layered in the co-extruded sheet 120 (eye insert). Hence, the monovalent alginate layer material feed 102 and the calcium chloride layer material feed 104 enter feedblock 110 separately and are selectively combined within feedblock 110. Once the sodium alginate layer material feed 102 and the calcium chloride layer material feed 104 are selectively layered and merged in feedblock 110, the combined melt stream 112 exits feedblock 110 and enters die 116 where the combined melt stream 112 is spread to the width of die 116 and emerges as co-extruded sheet 120. The co-extruded sheet 120 may then be allowed to cool.

FIG. 2 shows manifold die co-extrusion 200, which is another suitable process for making a co-extruded sheet 220 according to the invention. These unique dies have a designated manifold for each melt stream, enabling polymers to spread across the flow channel width independently. Each manifold can be tailored to a specific polymer. Multi-manifold dies are typically required to process coextruded structures where individual polymer layers have large viscoelastic differences. sodium alginate layer material feed 202 from main extruder 206 and calcium chloride layer material feed 204 from co-extruder 208 enter multi-manifold extrusion die 218 where they are aligned before being co-extruded as a co-extruded sheet 220 (eye insert).

Each of the main extruder 106, 206 and co-extruder 108, 208 converts the material fed into them into molten polymer, separately. The melt streams are then combined in either the feedblock 110 or the extrusion die 218.

The co-extruded sheets (eye inserts) described below may be produced by method 100, method 200 or by another co-extrusion method selected by a person of skill in the art. Where the co-extruded sheet of the invention has more than two layers it is understood that an appropriate number of extra co-extruders may be used, for example, a three layered co-extruded sheet could be made using one main extruder and two co-extruders or splitting the melt from either the main or co-extruder into two or more layers.

Main extruder 106, 206 is usually the largest extruder and has the highest throughput rate compared to co-extruders 108, 208. Therefore, for example, in a two-layered sheet configuration, the sodium alginate layer material feed 102, which is to comprise the relatively thick layer, is fed into the main extruder 106, 206. The eye insert is prepared by coextrusion process with polymers are extruded under 250 degree Celsius, preferred under 200 degrees Celsius, more preferred about 150 degree Celsius to avoid degradation of polymers ingredients.

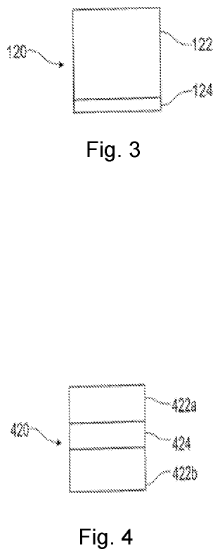

FIG. 3 shows one embodiment of a co-extruded sheet 120 according to the invention. Co-extruded sheet 120 has a sodium alginate layer 122 and a calcium chloride layer 124. According to this embodiment, the two-layer polymeric eye insert has a first monovalent alginate and a multivalent salt layer. The first monovalent alginate comprising monovalent alginate, and one or more mucoadhesive polymer. The multivalent salt layer comprising the multivalent salt, and the one or more humectant.

According to this embodiment, the first monovalent alginate may further comprise the one or more humectant; the multi valent salt layer may further comprising one or more mucoadhesive polymer.

According to this embodiment, the monovalent alginate is a sodium alginate, the multivalent salt is calcium chloride, the one or more humectant is selected from PEG, PVP or combinations thereof, the one or more mucoadhesive polymer is selected from HP guar, sodium hyaluronate or combinations thereof.

FIG. 4 shows another embodiment of a co-extruded sheet 420 according to the invention in which a multivalent salt layer 424 is located between, or is sandwiched between, a first monovalent alginate layer 422a and a second monovalent alginate 422b. According to this three-layer structure embodiment, the first monovalent alginate comprising monovalent alginate, and one or more mucoadhesive polymer. The multivalent salt layer comprising the multivalent salt, and the one or more humectant. The second monovalent alginate comprising monovalent alginate, and one or more mucoadhesive polymer. According to this three-layer structure embodiment, the first monovalent alginate may further comprise the one or more humectant; the second monovalent alginate may further comprise the one or more humectant; the multivalent salt layer, may further comprise one or more mucoadhesive polymer.

According to this three-layer structure embodiment, the monovalent alginate is a sodium alginate, the multivalent salt is calcium chloride, the one or more humectant is selected from PEG, PVP or combinations thereof, the one or more mucoadhesive polymer is selected from HP guar, sodium hyaluronate or combinations thereof. The first monovalent alginate layer and the second monovalent alginate layer have a same composition or a different composition. The same composition means not only the kind of ingredient but also the amount of ingredient is the same; otherwise, the compositions are different, for example first monovalent alginate layer and the second monovalent alginate layer have a different composition, even if both have the same ingredients but both have a different concentration of the same ingredients.

As previously discussed, in vivo studies indicate that traditional topical ophthalmic lubricants do not remain in the eye longer than approximately 25 minutes. However, use of one or more mucoadhesive polymers combined with at least one humectant, such as HP guar and hyaluronic acid blended with at least one humectant (such as PEG, PVP or combinations thereof), as well as a gelling agent (such as sodium alginate) may provide flexible films with tunable hydration and dissolution rates as needed even after the eye insert has been made for comfortable insertion or drug delivery. This can be accomplished by contacting the already made eye insert with a different concentration of divalent metal ion solution. Thereby the already made eye insert contacts with a higher concentration of divalent metal ion solution has a longer dissolution time.

While certain embodiments of the present invention are polymeric eye inserts containing a blend of hyaluronic acid, HP guar and PEG, it should be appreciated that other blends may be employed for polymeric eye inserts according to other embodiments of the present disclosure.

The eye inserts of the present disclosure are a platform to deliver lubricants and other pharmaceutically active agents to treat ocular surface symptoms (such as redness, itching and dryness). In some embodiments, the polymeric eye inserts can be used to prolong exposure of pharmaceutically active agents or provide extended drug delivery of pharmaceutically active agents to the eye. Thus, in some embodiments, the present disclosure provides a method of providing extended drug delivery or prolonging exposure of a pharmaceutically active agent to the eye, by administering a polymeric eye insert including the pharmaceutically active agent to a patient in need thereof.

In some embodiments, the present disclosure provides a method of treating or reducing the signs and/or symptoms of dry eye disease (keratoconjunctivitis sicca), comprising administering a polymeric eye insert according to the present disclosure to a patient in need thereof.

The following non-limiting Examples are provided to illustrate embodiments of the invention.

### EXAMPLES

### Example 1—Polymeric Eye Insert Dissolution Time Measurement

The dissolution time is measured by the procedure described here: 6 mm diameter and 50 microns thickness ocular insert film disks were cut.

The first step: the ocular insert film disks were activated by hydrating the ocular insert with 20 μL specified concentration of calcium chloride solution (hydrating solution or activating solution).

The second step: wait 2 minutes for equilibration, remove excess solution and placed at the bottom of the well. Adding 2 ml of immersion solution (PBS or isotonic saline with methyl blue) to the well. The well was placed in orbital shaker and shaken until the insert had dissolved (complete disintegration by visual inspection. The dissolution time was recorded.

1. A polymeric eye insert, the insert comprising:
a) 5% to 50% w/w gelling agent monovalent alginate, b) one or more humectant, c) one or more mucoadhesive polymers that are biocompatible with the ocular surface and tear film of the eye and d) 0.1% to 5% w/w multivalent salt, wherein the insert is prepared by a co-extrusion process, wherein polymeric eye insert comprising at least one monovalent alginate layer and at least one multivalent salt layer, wherein the at least one monovalent alginate layer is substantially free of the multivalent salt, wherein the at least multivalent salt layer is substantially free of the monovalent alginate.

2. The polymeric eye insert of claim 1, wherein the monovalent alginate is sodium alginate, potassium alginate, lithium alginate or ammonium alginate.

3. The polymeric eye insert of claim 1, wherein the multivalent salt is a salt containing Ca²⁺, Fe²⁺, Fe³⁺, Ag²⁺, Ag³⁺, Au²⁺, Au³⁺, Mg²⁺, Cu²⁺, Cu³⁺, Zn²⁺ or ammonium.

4. The polymeric eye insert of claim 2, wherein the monovalent alginate is sodium alginate and is present in an amount of from 15% to 30% w/w.

5. The polymeric eye insert of claim 4, wherein the sodium alginate has a weight molecular weight of 1 M to 2.5M Daltons.

6. The polymeric eye insert of claim 1, wherein the one or more mucoadhesive polymers are selected from the group consisting of: hyaluronic acid or salts thereof, hydroxypropyl methylcellulose (HPMC), methylcellulose, tamarind seed polysaccharide (TSP), guar, hydroxypropyl guar (HP guar), scleroglucan poloxamer, poly(galacturonic) acid, pectin, xanthan gum, xyloglucan gum, chitosan, sodium carboxymethylcellulose, polyvinyl alcohol, carbomer, polyacrylic acid and combinations thereof.

7. The polymeric eye insert of claim 6, wherein the one or more mucoadhesive polymers are HP guar, hyaluronic acid, or sodium hyaluronate and combinations thereof.

8. The polymeric eye insert of claim 7, wherein said HP guar has a weight average molecular weight of 1 to 3 million Daltons and said sodium hyaluronate has a weight average molecular weight of 0.1 to 3 million Daltons.

9. The polymeric eye insert of claim 7, wherein the one or more mucoadhesive polymers are present in an amount of from about 20% to about 90% w/w, about 50% to about 80% w/w, about 60% to about 70% w/w, or about 50% to about 60% w/w of the polymeric eye insert, provided that the sum of the % w/w of mucoadhesive polymers, % w/w of the sodium alginate, % w/w of the humectant agents and other components may not listed above is 100% w/w.

10. The polymeric eye insert of claim 9, wherein the one or more humectant is selected from the group consisting of: polyethylene glycol (PEG), a PEG derivative, PVP, Vitamin E, triethyl citrate and combinations thereof.

11. The polymeric eye insert of claim 10, wherein the humectant agent is present in an amount of from about 1% to about 10% w/w, about 1.5% to about 5% w/w, about 1.75% to about 3% w/w, or about 2% to about 2.5% w/w of the polymeric eye insert, provided that the sum of the % w/w of mucoadhesive polymers and % w/w of the biodegradable polymer, % w/w of the humectant agent and other components not listed above is 100% w/w.

12. The polymeric eye insert of claim 11, wherein the insert is comprised of approximately 40% to 48 w/w HP guar, approximately 20% to 40 w/w sodium hyaluronate, approximately 2% w/w PVP, approximately 15-30% w/w sodium alginate, and approximately 1% w/w PEG, provided that the sum of the % w/w of mucoadhesive polymer, % w/w of the sodium alginate, % w/w of the humectant agent and other components may not listed above is 100% w/w.

13. The polymeric eye insert of claim 1, wherein the polymeric eye insert has a multi-layered structure and comprises a first monovalent alginate and a multivalent salt layer, wherein a first monovalent alginate comprising monovalent alginate, and one or more mucoadhesive polymer, wherein the multivalent salt layer comprising the multivalent salt, and the one or more humectant.

14. The polymeric eye insert of claim 13, wherein the first monovalent alginate further comprises the one or more humectant.

15. The polymeric eye insert of claim 14, wherein the multivalent salt layer further comprises the one or more mucoadhesive polymer.

16. The polymeric eye insert of claim 13, wherein the monovalent alginate is a sodium alginate, the multivalent salt is calcium chloride, the one or more humectant is selected from PEG, PVP or combinations thereof, the one or more mucoadhesive polymer is selected from HP guar, sodium hyaluronate or combinations thereof.

17. The polymeric eye insert of claim 13, further comprising a second monovalent alginate layer, wherein the multivalent salt layer is sandwiched between the first and the second monovalent alginate layers, wherein the second monovalent alginate layer comprising the monovalent alginate and one or more mucoadhesive polymer, wherein the first monovalent alginate layer and the second monovalent alginate layer have a same composition or a different composition.

18. The polymeric eye insert of claim 17, wherein the second monovalent alginate further comprises the one or more humectant.

19. The polymeric eye insert of claim 16, wherein the monovalent alginate is a sodium alginate, the multivalent salt is calcium chloride, the one or more humectant is selected from PEG, PVP or combinations thereof, the one or more mucoadhesive polymer is selected from HP guar, sodium hyaluronate or combinations thereof.

20. The polymeric eye insert of claim 1, wherein the insert shape is a film, a rod, a sphere, an annulus, or a contact lens.
